(19) United States
(12) Patent Application Publication
(10) Pub. No.: US 2008/0082088 A1
(43) Pub. Date: Apr. 3, 2008

(54) SYSTEM AND METHOD FOR RESECTING CORNEAL TISSUE

(75) Inventors: Ronald M. Kurtz, Irvine, CA (US); Gagik Jotyan, Dorog (HU); Tibor Juhasz, Irvine, CA (US); Melvin A. Sarayba, Ladera Ranch, CA (US)

Correspondence Address: Towsend and Towsend and Crew LLP/IntraLase Corp., Two Embarcadero Center, Eighth Floor, San Francisco, CA, 94111, US

(73) Assignee: INTRALASE CORP.

(21) Appl. No.: 11/469,941

(22) Filed: Sep. 5, 2006

(51) Int. Cl. A61B 18/18

(52) U.S. Cl. 606/5

(57) ABSTRACT

A system and method for resecting and transplanting corneal tissue is disclosed. In a recipient cornea, a resection depth from the anterior surface of the recipient cornea is determined based upon a biomechanical model of the recipient cornea. A resection incision for resecting a posterior portion of the recipient cornea is made at the resection depth. Preferably, the incision is made using a surgical laser. Optionally, a contact lens may be placed against the anterior surface of the recipient cornea, wherein the shape of the anterior surface is conformed to the shape of the contact lens.

### BACKGROUND OF THE INVENTION

1. Field of the Invention

The field of the present invention is systems and procedures for transplanting corneas.

2. Background

Many different diseases or conditions of the cornea exist which completely or effectively rob those who suffer from such diseases or conditions of vision. Fortunately, corneal transplant procedures, which are becoming more commonplace, are capable of substantially restoring lost vision. Currently, there are many techniques and tools for performing corneal transplantation. Unfortunately, all suffer from inherent limitations or risk of complications.

A procedure called posterior lamellar keratoplasty (PLK), or deep lamellar keratoplasty, was previously developed to address these shortcomings for patients with endothelial cell dysfunction. In this procedure, a thin posterior or lenticule of stromal tissue (along with descemets membrane and endothelial cells attached) is removed from the cornea of a diseased eye. A similar procedure is performed on a donor eye to obtain donor tissue. When the donor tissue is placed in the recipient's eye, no sutures are required because the anterior surface of the recipients' cornea was left intact. One advantage of this procedure is that it results in little post-operative astigmatism. While PLK has its advantages, in practice it is difficult to perform, regardless of whether manual or automated instruments are used.

A procedure called descemets strip endokeratoplasty was introduced in attempts have been made to improve on PLK. In this procedure, the stromal tissue is not removed from the recipient's cornea, rather only the descemets membrane and endothelial cells are removed. While this simplifies the procedure, it results in an abnormally thick post-operative cornea and may degrade optical quality. In addition, there is the added risk of the additional tissue becoming dislodged from the recipient's cornea since no cavity was created into which it can be fully inserted.

Other attempts have been bade to improve on PLK using femtosecond lasers to incise both the recipient's and donor's cornea with high precision. While this makes PLK more practical, it also introduces some new difficulties into the procedure. Specifically, contact lenses, both flat and curved, are generally used with femtosecond lasers in order to provide proper registration of the cornea with the focal point of the pulsed laser beam. These contact lenses distort the shape of the cornea by forcing the anterior surface of the cornea to conform to the curvature of the lens. This distortion of the cornea introduces folds or wrinkles into the corneal tissue, with the folds being more pronounced in the posterior portion of the cornea. Thus, when a resection incision is made, the folds or wrinkles can cause the incision to have an irregular surface when the cornea returns to its natural curvature if the incision is made too near the posterior surface of the cornea. Such an irregular surface, however, can have adverse effects on wound healing and optical quality of the post-operative cornea.

Another problem arises when the resection incision is made too near the anterior surface of the cornea. Under such circumstances, the surgically repaired cornea may begin to weaken and deteriorate due to ectasia because the anterior portion was overly thinned during the procedure.

### SUMMARY OF THE INVENTION

The present invention is directed toward a system and method for resecting corneal tissue. In the system, a surgical laser emits a pulsed laser beam which is directed into the cornea by a focusing assembly. An interface is programmed with a biomechanical model of corneal tissue, which is used to provide a depth range for selection of a resection depth from an anterior corneal surface. The selected resection depth is received by a controller which employs the focusing assembly to move the focal point of the pulsed laser beam and make a resection incision at the resection depth for resecting a portion of the cornea.

In the method, the resection depth is first determined using a biomechanical model of the cornea. Thereafter, a resection incision is made at the resection depth for purposes of resecting a posterior portion of the cornea.

Other options may be added to the above system or process, either singly or in combination. In a first option, the resection depth is determined using the anterior surface of the cornea as a reference. In a second option, a contact lens is placed against the anterior surface of the cornea. The contact lens is adapted to conform the anterior surface of the cornea to the shape of the contact lens, which preferably has a radial or planar form. With the contact lens in place, the resection incision is made using a surgical laser. In a third option, the resection depth is selected to minimize surface irregularities resulting from the resection incision. In a fourth option, the resection depth is selected to minimize post-operative weakening of the cornea. In a fifth option, the resection incision is placed at a uniform distance from one of the posterior surface of the cornea or the anterior surface of the cornea. In a sixth option, a posterior portion of corneal tissue is resected from a donor cornea, and this donor tissue is grafted into the recipient cornea. In resecting the donor tissue, the ratio of the depth of the resection incision in the recipient cornea, as compared to the overall thickness of the recipient cornea, is first determined. From this ratio, the depth of the resection incision in the donor cornea may be determined. This is done by making the depth of the resection incision in the donor cornea have the same ratio as compared to the overall thickness of the donor cornea.

Accordingly, an improved system and method of resecting corneal tissue are disclosed. Advantages of the improvements will appear from the drawings and the description of the preferred embodiment.

### BRIEF DESCRIPTION OF THE DRAWINGS

In the drawings, wherein like reference numerals refer to similar components:

FIG. 1 is a flow chart illustrating steps for performing a corneal transplant procedure;

FIGS. 2A & 2B illustrate resection incisions in a recipient cornea and a donor cornea, respectively, as part of a corneal transplant procedure; and

FIG. 3 is a schematic view of a system for resecting corneal tissue.

### DETAILED DESCRIPTION OF THE PREFERRED EMBODIMENTS

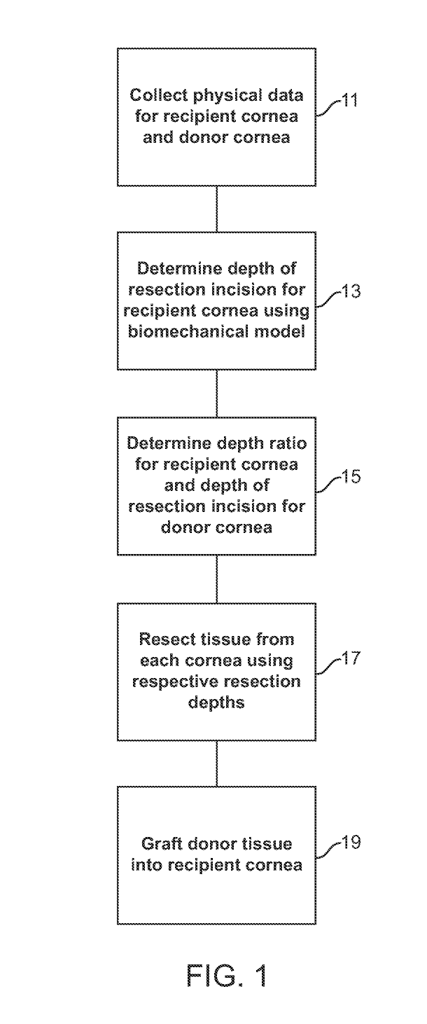

Turning in detail to the drawings, FIG. 1 is a flow chart illustrating a process for performing a PLK procedure. The basic steps of the PLK procedure are (1) collect physical data 11 for both the recipient cornea and the donor cornea; (2) determine the depth of the resection incision 13 for the recipient cornea; (3) determine the depth ratio of the resection incision 15 in the recipient cornea and the depth of the resection incision in the donor cornea; (4) resect tissue 17 from both the recipient and donor corneas; and (5) graft 19 the donor tissue into the recipient cornea. Each step is explained in further detail below.

The physical data collected includes thickness measurements of both the recipient and donor corneas. These thickness measurements are used to develop a thickness profile of each cornea. Additional physical data may also be collected for each cornea, with the type of data collected being dependent upon the requirements of biomechanical model used in the subsequent step of the PLK procedure. This thickness profile, along with any other data needed for the biomechanical model, may be obtained by any one of the many known methods for measuring the physical structure of the eye, with the preferred method being through optical coherence tomography (OCT). Many commercially available OCT scanners are capable of performing such measurements. One example is the Visante™ OCT scanning system, manufactured by Carl Zeiss Meditec, which has an office in Dublin, Calif. One advantage of the Visante™ OCT system is that it does not make contact with the cornea when performing the OCT scan.

Following collection of the physical data, the depth of the resection incision for the recipient cornea is determined using the biomechanical model. Preferably, the biomechanical model takes into account stresses introduced into the corneal tissue when the cornea is conformed to the shape of a contact lens set against the anterior surface when the resection incision is made with a surgical laser system. Such stresses can cause folds in the corneal tissue which are more pronounced in the posterior regions of the cornea. A resection incision made in areas with more pronounced folds can lead directly to surface irregularities at the resection incision when contact lens is removed and the cornea returns to its normal shape. Therefore, the biomechanical model is used to place the resection incision at a depth which minimizes or eliminates surface irregularities at the resection incision.

The biomechanical model also preferably takes into account the post-operative stability of the recipient cornea when determining the depth of the resection incision. Without sufficient post-operative stability, the cornea may undergo weakening through pathological deterioration, e.g., ectasia, thereby exposing the recipient to a risk of vision impairment. Maintaining sufficient tissue between the anterior surface of the recipient cornea and the resection incision is an important factor to maintaining post-operative stability. The biomechanical model is therefore employed to determine the minimum depth from the anterior surface at which the resection incision is likely to cause post-operative stability problems.

Optionally, the biomechanical model may be developed to employ a database of corneal measurements collected from many patients. Using information from the database, together with at least the known curvature of the contact lens, such a biomechanical model could be used to determine an appropriate depth for the resection incision.

The resection depth selected for the recipient cornea is selected to be between the minimum depth needed to create post-operative stability in the recipient cornea and an appropriate depth to avoid or eliminate surface irregularities at the resection incision. In the event that there is no region between the two extremes, maintaining post-operative stability in the cornea is preferred. In such instances, the radius of the contact lens upon which the shape of the cornea is conformed for purposes of the PLK procedure and the biomechanical model could be enlarged to reduce stresses on the corneal tissue and help create a region in which post-operative stability can be maintained along with minimizing irregularities at the resection incision.

The biomechanical model may be one of several previously developed models which are known to skilled artisans, or it may be one which is specifically developed for the PLK procedure. Modeling software, such as the software published by Structural Research & Analysis Corp. of Santa Monica, Calif. under the title “Cosmos/M”, may be used to develop biomechanical models. One such model is described in the article by G. Djotyan et al., “Finite Element Modeling of Posterior Lamellar Keratoplasty: Construction of Theoretical Nomograms for Induced Refractive Errors”, Ophthalmic Research, Vol. 38, n.5, 2006.

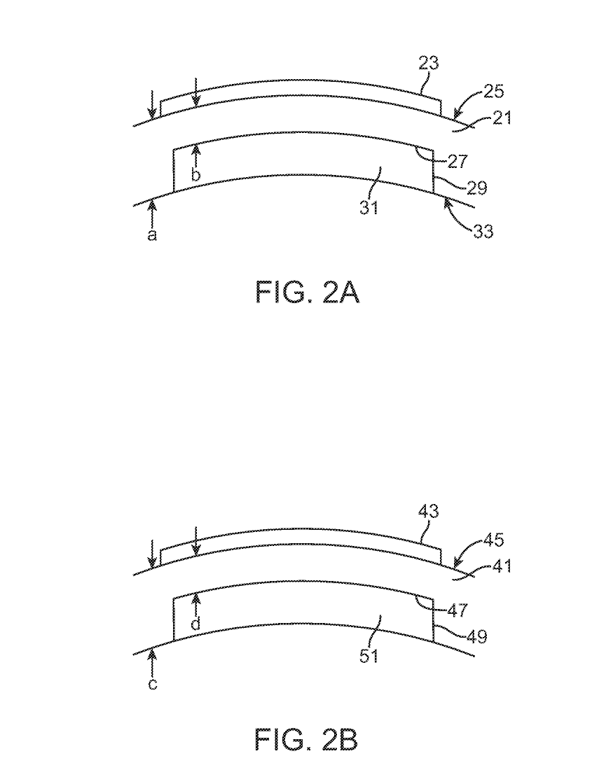

FIG. 2A illustrates the recipient cornea 21 with a contact lens 23 placed against the anterior surface 25 of the recipient cornea 21. The resection incision 27 and the sidecut 29 are made to remove a posterior portion of corneal tissue 31 in preparation for the transplant. The resection incision 27 may be made using several different techniques. However, it should be noted that not all techniques provide the same quality of clinical results following the transplant. One technique is to make the resection incision 27 at a uniform distance from the posterior surface 33 of the cornea. U.S. patent application Ser. No. 11/375,542, the disclosure of which is incorporated herein by reference, discloses a method of making such an incision. By way of further example, techniques for making the resection incision 27 at a uniform distance from the anterior surface 25 are disclosed in U.S. Pat. No. 5,993,438, U.S. Pat. No. 6,730,074, and U.S. Patent Publication No. 20050245915. Other techniques known to skilled artisans may also be employed.

The thickness of the recipient cornea 21 is expressed as ‘a’. The depth, b, of the resection incision 27 shown in FIG. 1 is measured from the anterior surface 25 of the cornea. The ratio of the depth, b, to the thickness, a, is employed to determine the depth of the resection incision in the donor cornea as described below.

Similar to the resection incision 27, the sidecut 29 may also be formed through the different techniques that are known to skilled artisans.

The contact lens 23 is a rigid lens placed against the anterior surface 25, thereby forcing the anterior surface 25 to conform to the shape of the contact lens 23. Preferably, the contact lens 23 conforms the anterior surface 25 to a radial or planar shape. U.S. Pat. No. 5,549,632, which is incorporated herein by reference, describes making a laser incision by deforming the shape of the cornea. U.S. Pat. No. 6,863,667 and U.S. patent application Ser. No. 11/258,399, both of which are incorporated herein by reference, describe patient interface devices which may be used to align the surgical laser with the recipient cornea for purposes of making accurate incisions. Of course, the contact lens is not needed if the surgical laser system is capable of alignment with sufficient precision without use of the contact lens.

FIG. 2B illustrates the donor cornea 41 with a contact lens 43 placed against the anterior surface 45 of the donor cornea 41. The resection incision 47 and the sidecut 49 are made to remove the donor tissue 51 for grafting into the recipient cornea. The resection incision 47 and the sidecut 49 are preferably made using the same techniques employed to incise the recipient cornea. The ratio of the depth, d, of the resection incision 47 to the thickness, c, of the donor cornea 41 is preferably the same as the ratio that was set for the recipient cornea. By setting the depth of the resection incision 47 in this manner, compensation is made in the event that the donor cornea 41 swells due to fluid absorption following removal from the donor eye. Such swelling occurs because the donor cornea is resected from the donor eye in facility separate from the one in which the transplant is performed.

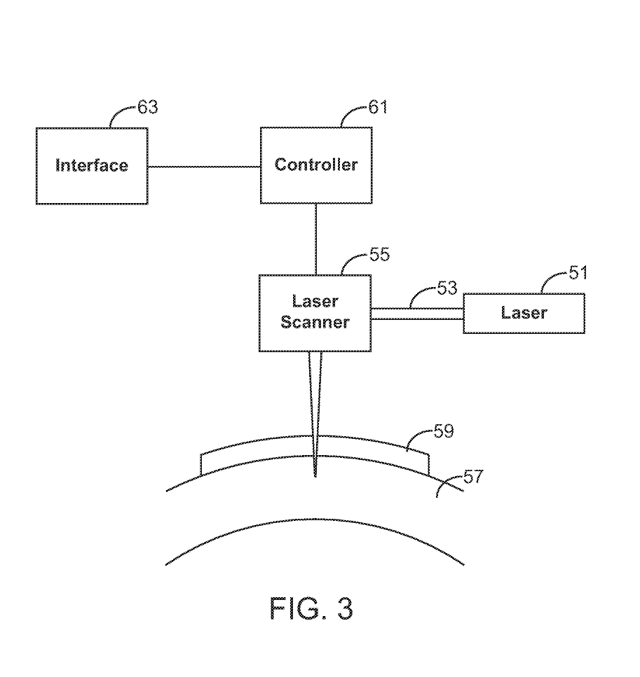

Referring to FIG. 3, a surgical system is shown which may be used to incise both a donor cornea or a recipient cornea. A femtosecond surgical laser 51 generates a pulsed laser beam 53 and directs that beam into the focusing assembly 55, which in turn focuses the pulsed beam 53 into the cornea 57. A contact lens 59 is placed over the cornea to deform the anterior corneal surface as described above. The controller 61 is a programmable computer which precisely controls the location of the beam focal point within the cornea 57 according to parameters received from the programmable surgeon interface 63. The interface 63 is programmed with a biomechanical model of the cornea and presents the surgeon with an incision depth range within which the surgeon may select the resection depth. The resection depth is received by the controller 61, which uses the focusing assembly 55 to make the resection incision at the resection depth.

A pulsed laser beam having ultra-short pulses, preferably in the femtosecond range, is employed to make the incisions. The laser may be of the type described in U.S. Pat. No. 4,764,930, producing an ultra-short pulsed beam as described in one or both of U.S. Pat. No. 5,984,916 and U.S. Patent No. RE37,585. The disclosures of the aforementioned patents are incorporated herein by reference in their entirety. Commercial lasers capable of performing the incisions are available from IntraLase Corp. of Irvine, Calif.

Thus, a system and method of resecting corneal tissue are disclosed. While embodiments of this invention have been shown and described, it will be apparent to those skilled in the art that many more modifications are possible without departing from the inventive concepts herein. The invention, therefore, is not to be restricted except in the spirit of the following claims.

What is claimed is:

1. A method of resecting corneal tissue, the method comprising:
determining a resection depth from an anterior surface of a cornea using a corneal biomechanical model; and
making a resection incision for resecting a posterior portion from the cornea, wherein the resection incision is made at the resection depth.

2. The method of claim 1, wherein making the resection incision includes making the resection incision using a surgical laser.

3. The method of claim 1 further comprising:
placing a contact lens against the anterior surface, the contact lens being adapted to conform the anterior surface to a shape of the contact lens; and
making the resection incision using a surgical laser.

4. The method of claim 3, wherein the contact lens applanates the anterior surface.

5. The method of claim 4, wherein the resection depth is selected to minimize surface irregularities at the resection incision.

6. The method of claim 1, wherein the resection depth is selected to minimize post-operative weakening of the cornea.

7. The method of claim 1, wherein the resection incision is at a uniform distance from one of a posterior surface of the cornea or the anterior surface.

8. A method of resecting corneal tissue, the method comprising:
determining a resection depth from an anterior surface of a cornea using a biomechanical model of the cornea; and
placing a contact lens against the anterior surface, the contact lens being adapted to conform the anterior surface to a shape of the contact lens; and
making a resection incision for resecting a posterior portion from the cornea, wherein the resection incision is made at the resection depth using a surgical laser, and the resection incision is at a uniform distance from one of a posterior surface of the cornea or the anterior surface.

9. The method of claim 8, wherein the contact lens applanates the anterior surface.

10. The method of claim 8, wherein the resection depth is selected to minimize surface irregularities at the resection incision.

11. The method of claim 8, wherein the resection depth is selected to minimize post-operative weakening of the cornea.

12. A method of transplanting corneal tissue from a donor cornea to a recipient cornea, the method comprising:
determining a recipient resection depth from a recipient anterior surface of the recipient cornea using a biomechanical model of the recipient cornea;
making a recipient resection incision for resecting a recipient posterior portion from the recipient cornea, wherein the recipient resection incision is made at the recipient resection depth;
making a donor resection incision for resecting a donor posterior portion from the donor cornea; and
grafting the donor posterior portion into the recipient cornea.

13. The method of claim 12, wherein at least one of the recipient resection incision or the donor resection incision is made using a surgical laser.

14. The method of claim 12 further comprising:
placing a contact lens against the recipient anterior surface, the contact lens being adapted to conform the recipient anterior surface to a shape of the contact lens; and
making the recipient resection incision using a surgical laser.

15. The method of claim 14, wherein the contact lens applanates the recipient anterior surface.

16. The method of claim 15, wherein the recipient resection depth is determined to minimize surface irregularities at the recipient resection incision.

17. The method of claim 12, wherein the recipient resection depth is determined to minimize post-operative weakening of the recipient cornea.

18. The method of claim 12, wherein the recipient resection incision is at a uniform distance from one of a recipient posterior surface of the recipient cornea or a recipient anterior surface.

19. The method of claim 12 further comprising:
determining a ratio of the recipient resection depth to a recipient thickness of the recipient cornea; and
making the donor resection incision at a donor resection depth from a donor anterior surface of the donor cornea, wherein the donor resection depth, as compared to a donor thickness of the donor cornea, is determined by the ratio.

20. The method of claim 19, wherein the donor resection incision is at a uniform distance from one of the donor posterior surface or a donor anterior surface of the recipient cornea.

21. A system for resecting corneal tissue, the system comprising:
an interface programmed with a biomechanical model of corneal tissue, the interface being adapted to provide a depth range using the biomechanical model for selection of a resection depth from an anterior corneal surface;
a surgical laser adapted to emit a pulsed laser beam;
a focusing assembly adapted to focus the pulsed laser beam into a cornea;
a controller adapted to receive the resection depth from the interface, to move a focal point of the pulsed laser beam within a cornea using the focusing assembly, and to direct the focal point of the pulsed laser beam to make a resection incision for resecting a portion of the cornea, wherein the resection incision is made at the resection depth.

22. The system of claim 21 further comprising:
a contact lens adapted to be placed against the anterior surface and conform the anterior surface to a shape of the contact lens, wherein the biomechanical model accounts for deformation of the cornea.

23. The system of claim 22, wherein the contact lens applanates the anterior surface.

24. The system of claim 21, wherein the depth range is provided to minimize surface irregularities at the resection incision.

25. The system of claim 21, wherein the depth range is provided to minimize post-operative weakening of the cornea.

26. The system of claim 21, wherein the resection incision is at a uniform distance from one of a posterior surface of the cornea or the anterior surface.

27. The system of claim 21, wherein the controller is adapted to make the resection incision for resecting a posterior portion of the cornea
